(19) United States
(12) Patent Application Publication
(10) Pub. No.: US 2019/0328269 A1
(43) Pub. Date: Oct. 31, 2019

(54) BODY ANALYZER

(71) Applicant: Xinqiang XU, SHENZHEN (CN)

(72) Inventor: Xinqiang XU, SHENZHEN (CN)

(21) Appl. No.: 16/226,562

(22) Filed: Dec. 19, 2018

(30) Foreign Application Priority Data
Apr. 27, 2018 (CN) 201810389943.6
Apr. 27, 2018 (CN) 201820624844.7
Jul. 27, 2018 (CN) 201810847162.7
Jul. 27, 2018 (CN) 201821214351.2

(51) Int. Cl. A61B 5/053; A61B 5/107; A61B 5/00

(52) U.S. Cl. CPC A61B 5/0537; A61B 5/1072; A61B 5/0022; A61B 5/4875; A61B 5/4872

(57) ABSTRACT

A body analyzer includes a main body including a platform contacting feet of a subject, an information transmitter, a weighing and body analyzing mechanism, a signal processor, and a wireless communication device, and a detecting board abutting against the subject's top head. At least one of the main body and the detecting board has a signal emitter, and at least one of the two has a signal receiver, thereby measuring a distance between the platform and the detecting board. The information transmitter emits data of the signal emitter and the signal receiver to the weighing and body analyzing mechanism. The weighing and body analyzing mechanism calculates body compositions of the subject. The signal processor processes data from the signal emitter, the signal receiver, and the weighing and body analyzing mechanism. The wireless communication device emits a processed data to an external mobile device or server via a wireless network.

### CROSS-REFERENCES TO RELATED APPLICATIONS

This application claims priority to Chinese Patent Application No. 2018103899436, entitled “BODY ANALYZER” filed Apr. 27, 2018, Chinese Patent Application No. 2018206248447, entitled “BODY ANALYZER” filed Apr. 27, 2018, Chinese Patent Application No. 2018108471627, entitled “BODY ANALYZER” filed Jul. 27, 2018, Chinese Patent Application No. 2018212143512, entitled “BODY ANALYZER” filed Jul. 27, 2018, the contents of which are expressly incorporated herein by reference in its entirety.

### FIELD

The present disclosure relates to household health care technologies, and more particularly relates to a body analyzer.

### BACKGROUND

With an improvement of people's living standards, a demand for health increases accordingly and body analyzer becomes a popular product. The body analyzer is a health care device that can measure weight, body fat, body water, body muscle, basal metabolism, body mass index, bone mass, and other compositions.

Currently, the conventional body analyzer cannot measure a height of a subject. Other height measuring instrument is required to measure the height of the subject, which brings inconvenience to the use thereof.

### SUMMARY

According to various embodiments of the present disclosure, a body analyzer is provided.

A body analyzer includes a main body and a detecting board. The main body includes a platform in contact with feet of a subject. The detecting board is separated from the main body and configured to abut against a top head of the subject. The detecting board is capable of moving towards or away from the platform. At least one of the main body and the detecting board is provided with a signal emitter, and at least one of the main body and the detecting board is provided with a signal receiver. The signal emitter and the signal receiver are used to measure a distance between the platform and the detecting board. The main body further includes an information transmitter, a weighing and body analyzing mechanism, a signal processor, and a wireless communication device. The information transmitter is configured to transmit data of the signal emitter and the signal receiver to the weighing and body analyzing mechanism. The weighing and body analyzing mechanism is used to calculate body compositions of the subject. The signal processor is configured to process data from the signal emitter, the signal receiver, and the weighing and body analyzing mechanism. The wireless communication device is used to transmit a processed data to an external mobile device or server via a wireless network.

A body analyzer includes a main body and a detecting board. The body analyzer includes a platform in contact with feet of a subject. The main body is provided with a signal emitter and a signal receiver. The detecting board is separated from the main body and configured to abut against a top head of the subject, and the detecting board is capable of moving towards or away from the platform. The detecting board, the signal emitter, and a signal receiver cooperatively measure a distance between the platform and the detecting board. The main body further includes a weighing and body analyzing mechanism, a signal processor, an information transmitter, a wireless communication device, and a display screen. The wireless communication device is used to perform a data interaction with an external mobile device or server via a wireless network. The information transmitter is used to transmit data of the signal emitter and the signal receiver to the weighing and body analyzing mechanism. The weighing and body analyzing mechanism is used to calculate body compositions of the subject. The signal processor is electrically connected to the weighing and body analyzing mechanism, the signal emitter, the signal receiver, the wireless communication, and the display screen respectively, and is used to process data of the signal emitter, the signal receiver, and the weighing and body analyzing mechanism. A processed data is sent to the display screen for displaying and is sent to the external mobile device or server via the wireless communication device.

The details of one or more implementations of the subject matter described in this specification are set forth in the accompanying drawings and the description below. Other potential features, aspects, and advantages of the subject matter will become apparent from the description, the drawings, and the claims.

### BRIEF DESCRIPTION OF THE DRAWINGS

To illustrate the technical solutions according to the embodiments of the present disclosure or in the prior art more clearly, the accompanying drawings for describing the embodiments or the prior art are introduced briefly in the following. Apparently, the accompanying drawings in the following description are only some embodiments of the present disclosure, and persons of ordinary skill in the art can derive other drawings from the accompanying drawings without creative efforts.

FIG. 1a is a top view of a main body of a body analyzer in accordance with a first embodiment.

FIG. 1b is a side view of the body analyzer in accordance with the first embodiment in a using status.

FIG. 2 is a block diagram of a body analyzer of FIG. 1b.

FIG. 3a is a top view of a main body of a body analyzer in accordance with a second embodiment.

FIG. 3b is a side view of the body analyzer in accordance with the second embodiment in a using status.

FIG. 4 is a block diagram of a body analyzer of FIG. 3b.

### DETAILED DESCRIPTION OF THE INVENTION

Reference will now be made to the drawings to describe, in detail, embodiments of the present camera module. It should be noted that references to “an” or “one” embodiment in this disclosure are not necessarily to the same embodiment, and such references mean at least one.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein”, “above”, “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list.

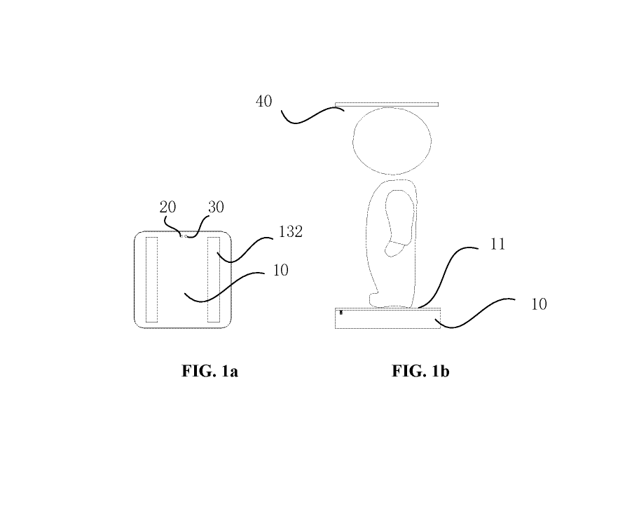

Referring to FIGS. 1a and 1b, a body analyzer 100 according to a first embodiment is provided. In this embodiment, the body analyzer 100 includes a main body 10 and a detecting board 40.

The main body 10 includes a platform 11, which can be in contact with feet of a subject, such as an adult, or a child. In one embodiment, the main body 10 is provided with a signal emitter 20 and a signal receiver 30. The signal emitter 20 is used to emit a signal for measuring a height of the subject. The signal emitter 20 can be at least one selected from a group consisting of a laser emitter, an LED emitter, an infrared emitter, an ultrasonic transmitter, an electromagnetic wave transmitter, and a millimeter wave transmitter. The signal receiver 30 is used to receive the height measuring signal reflected by the detecting board 40.

The detecting board 40 is separated from the main body 10 and is used to abut against a top head of the subject. The detecting board 40 can be moved towards or away from the platform 11. When abutting against the top head of the subject, the detecting board 40 can reflect the signal emitted from the signal emitter 20 to the signal receiver 30. The detecting board 40 can be any planar board that can achieve a signal reflection, such as a plastic plate or even a cell phone.

When measuring, the subject steps on the platform 11 and holds the detecting board 40 on the top head thereof. The signal emitter 20 emits the height measuring signal towards the detecting board 40. The height measuring signal is reflected by the detecting board 40 and is received by the signal receiver 30, thereby achieving the height measuring of the subject.

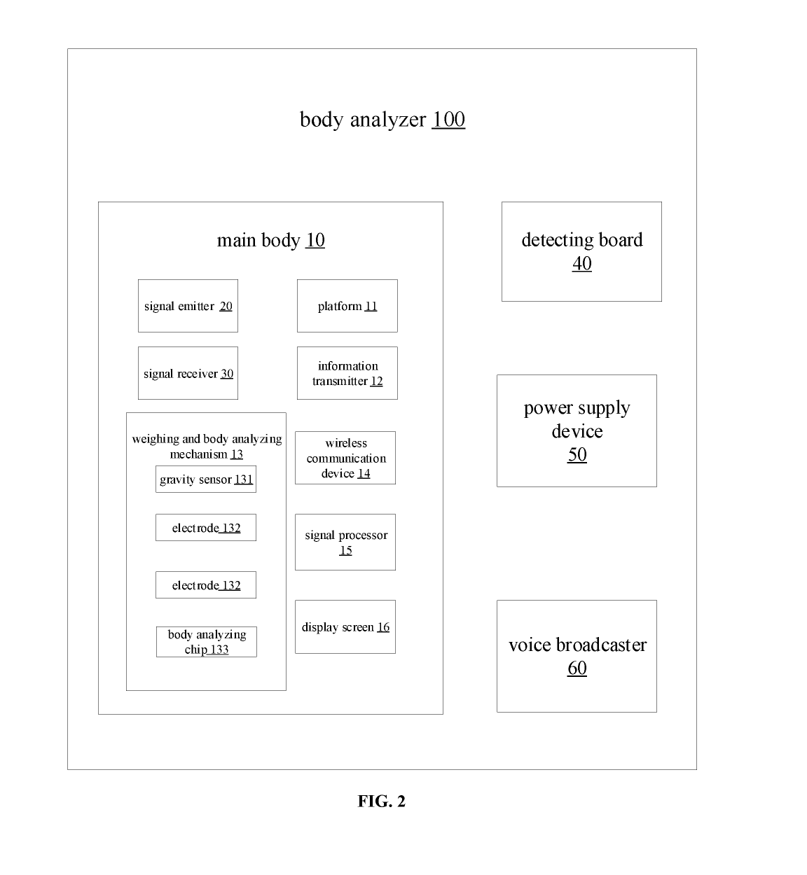

Referring to FIG. 2, the main body 10 further includes an information transmitter 12, a weighing and body analyzing mechanism 13, a wireless communication device 14, a signal processor 15, and a display screen 16.

The information transmitter 12 is electrically connected to the signal emitter 20, the signal receiver 30, and the weighing and body analyzing mechanism 13, respectively. The information transmitter 12 is used to transmit data of the signal emitter 20 and the signal receiver 30 to the weighing and body analyzing mechanism 13.

The weighing and body analyzing mechanism 13 receives the data transmitted by the information transmitter 12 and calculates body compositions. It should be understood that the data transmitted by the information transmitter 12 and received by the weighing and body analyzing mechanism 13 can be used in the calculation or determination of the body compositions, or the body compositions can be calculated or determined without the data transmitted by the information transmitter 12 and received by the weighing and body analyzing mechanism 13. In one embodiment, the weighing and body analyzing mechanism 13 can calculate or determine body compositions based on bioelectrical impedance analysis (BIA) technology. The body compositions can be at least one selected from a group consisting of weight, body fat, body water, body muscle, basal metabolism, body mass index, bone mass, and other compositions. In one embodiment, the weighing and body analyzing mechanism 13 includes a gravity sensor 131, two electrodes 132, and a body analyzing chip 133. The detail measurement processes of the body compositions such as body fat, body water, body muscle, basal metabolism, body mass index, and bone mass can be found in Chinese Patent Applications NO. CN205317331U, and CN204909395U, the disclosure content of which are incorporated herein by reference in its entirety. It should be understood that in other embodiments, the number of the electrodes can be 4, 6, and other quantities.

Body Analyzer that utilizes a “foot-to-foot” BIA (bioelectrical impedance analysis) technology to determine internal body composition, which is used to measure a weight and body compositions of the subject.

The wireless communication device 14 is used to interact with an external mobile device or server via a wireless network. The wireless communication device can be at least one selected from a group consisting of a Wi-Fi device, a Bluetooth device, a ZigBee device, a Zwave device, a WLAN device, a GSM device, a 2G device, a 3G device, a 4G device, and a 5G devices.

The signal processor 15 is electrically connected to the signal emitter 20, the signal receiver 30, the weighing and body analyzing mechanism 13, the wireless communication device 14, and the display screen 16, respectively. The signal processor 15 can be a microprocessor, or other chips that can implement data processing, etc. The signal processor 15 processes data transmitted from the signal emitter 20 and the signal receiver 30 to obtain height data of the subject, processes data transmitted by the weighing and body analyzing mechanism 13 to obtain weight and body compositions data of the subject, and transmits the height data and the weight and body compositions data to the display screen 16. The signal processor 15 further transmits the height data and the weight and body compositions data to the external mobile device or server via the wireless communication device 14. The external mobile device or server can store the height data and the weight and body compositions data, or display a constitutional index of the subject via a certain manner (such as curve diagram, etc.), thereby establishing a relatively comprehensive human health index of the subject and making it convenient for a user to have an intuitive understanding of the constitutional index of the subject.

The display screen 16 receives and displays the height data and the weight and body compositions data transmitted by the signal processor 15. The display screen 15 can be an LCD or a digital display screen.

In this embodiment, the body analyzer 100 further includes a power supply device 50. The power supply device 50 is configured to supply power to the body analyzer 100. The power supply device 50 can be a battery or other rechargeable device. In one embodiment, the power supply device can convert an external alternating current into a direct current to supply power to the body analyzer 100.

In this embodiment, the body analyzer 100 further includes an acoustic indicator device 60 electrically connected to the signal processor 15. The acoustic indicator device 60 is used to broadcast measuring results transmitted by the signal processor 15 in voice.

The aforementioned body analyzer 100 includes the main body 10 and the detecting board 40 separated from the main body 10. The main body 10 includes the signal emitter 20, the signal receiver 30, the information transmitter 12, the weighing and body analyzing mechanism 13, the wireless communication device 14, the signal processor 15, and the display screen 16. The signal emitter 20 emits a height measuring signal. The height measuring signal is received by the signal receiver 30 after being reflected by the detecting board 40. A measured distance between the platform 11 and the detecting board 40 is actually the height of the subject. The wireless communication device 14 interacts with the external mobile device or server via the wireless network. The signal processor 15 is electrically connected to the signal emitter 20, the signal receiver 30, the information transmitter 12, the weighing and body analyzing mechanism 13, the wireless communication device 14 and the display screen 16, respectively. The signal processor 15 processes the data of the signal emitter 20, the signal receiver 30, the weighing and body analyzing mechanism 13, and transmits the processed data to the display screen 16 for display and transmits the processed data to the external mobile device or server via the wireless communication device 14. In this way, the accurate weight and height data of the subject can be obtain via the processing of the signal processor 15 on the received signal, which avoids manually height measurement via mechanical means, and ensures an accuracy of a measuring result. The measuring result can be visually displayed on the display screen and can be transmitted to the external mobile device or server for storage via the wireless communication device, thereby avoiding manually record of the measuring result, which saves time and labor, and is convenient and practical.

In an alternative embodiment, the main body 10 can be further provided with an operation button (not shown). The operation button can be at least one selected from a group consisting of a lock button, an acoustic indicating button, a wireless transmission button, a power button, and a height display button. The lock button is used to prevent display values of the weight and body compositions data from frequently changing due to shaking of the subject on the body analyzer 100. When the subject presses the lock button, the display values of the weight and body compositions data thereof are no longer changed. The acoustic indicating button is used to provide a choice for the subject about whether broadcasting the measuring results (the height, weight, and body compositions data, etc.) thereof in voice or not. When the acoustic indicating button is pressed, the body analyzer 100 broadcasts the measuring results, otherwise the broadcasting is not performed. The wireless transmission button is used to provide a choice for the subject about whether transmitting the measuring results to the external mobile device or server via the wireless network. When the wireless transmission button is pressed, the body analyzer 100 transmits the measuring results to the external mobile device or server, otherwise the transmission of the measuring results is not performed. The power button is used to control whether energizing the body analyzer 100 or not. When the power button is pressed, the body analyzer 100 is powered on, otherwise the body analyzer 100 is not powered. The height display button is used to provide a choice for the subject about whether measuring and displaying the height data thereof. A default state of the height display button indicates to measure and display the height data. When the height display button is pressed, the height measurement and display are not performed.

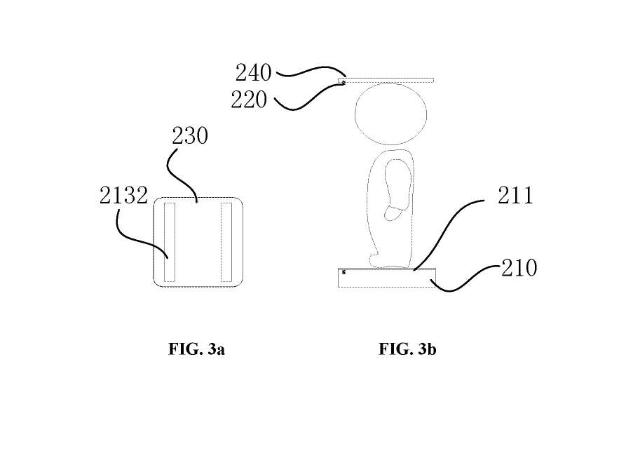

Referring to FIGS. 3a to 3b, a body analyzer 200 according to a second embodiment is provided. The body analyzer 200 is similar to the body analyzer 100.

Correspondingly, in this embodiment, the body analyzer 200 includes a main body 210 and a detecting board 240. The main body 210 includes a platform 211, which can be in contact with feet of a subject. The detecting board 40 is separated from the main body 10, and is used to abut against a top head of the subject. A difference between the body analyzer 200 of this embodiment and the body analyzer 100 of the first embodiment is that the detecting board 240 is provided with a signal emitter 220, and the main body 210 is provided with a signal receiver 230. The signal emitter 220 is used to emit a signal for measuring a height of the subject. The signal receiver 230 is used to receive the height measuring signal transmitted by the signal emitter 220. When measuring, the subject steps on the platform 211 and holds the detecting board 240 abutting against the top head thereof, so that the signal transmitted by the signal emitter 220 can be received by the signal receiver 230.

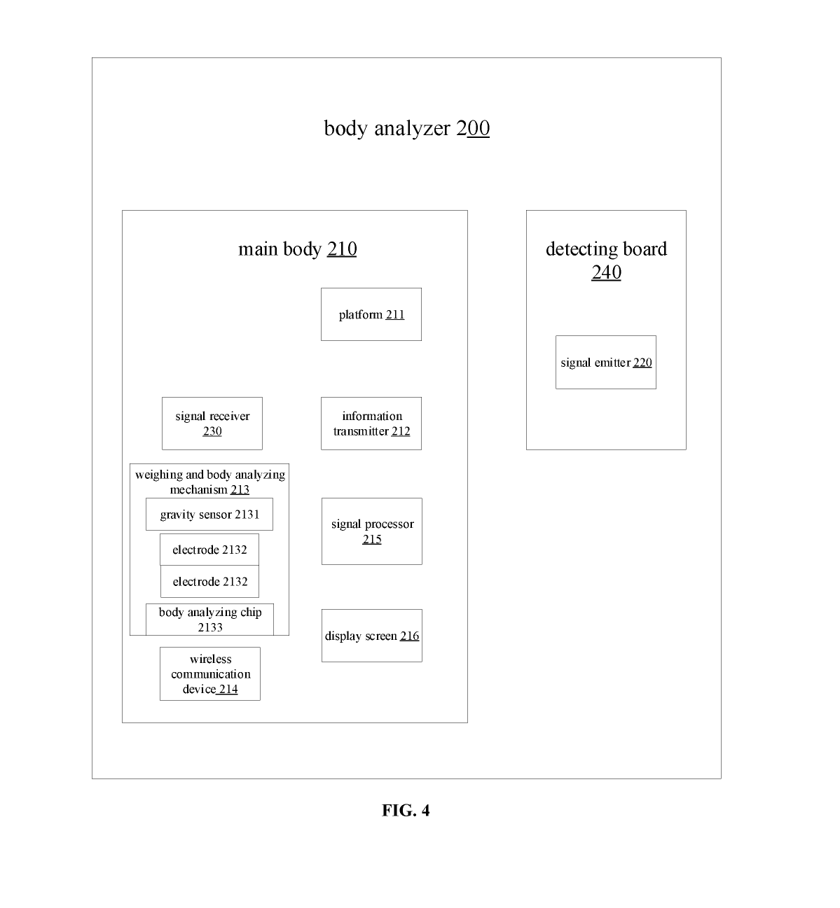

Referring to FIG. 4, the body analyzer 210 further includes an information transmitter 212, a weighing and bodying analyzing mechanism 213, a wireless communication device 214, a signal processor 215, and a display screen 216.

The information transmitter 212 is electrically connected to the signal receiver 230, and the weighing and body analyzing mechanism 213, respectively. The information transmitter 212 is used to transmit data received by the signal receiver 230 to the weighing and body analyzing mechanism 213.

The weighing and body analyzing mechanism 213 receives the data transmitted by the information transmitter 212. The weighing and body analyzing mechanism 213 is used to calculate body compositions. In one embodiment, the weighing and body analyzing mechanism 213 can calculate or determine body compositions based on bioelectrical impedance analysis (BIA) technology. The body compositions can be at least one selected from a group consisting of weight, body fat, body water, body muscle, basal metabolism, body mass index, bone mass, and other compositions. In one embodiment, the weighing and body analyzing mechanism 213 includes a gravity sensor 2131, two electrodes 2132, and a body analyzing chip 2133. The detail measurement processes of the body compositions such as body fat, body water, body muscle, basal metabolism, body mass index, and bone mass can be found in Chinese Patent Applications NO. CN205317331U, and CN204909395U, the disclosure content of which are incorporated herein by reference in its entirety.

The wireless communication device 214 is used to interact with an external mobile device or server via a wireless network.

The signal processor 215 is electrically connected to the signal receiver 230, the weighing and body analyzing mechanism 213, the wireless communication device 214, and the display screen 216, respectively. The signal processor 215 processes data transmitted of the signal emitter 220 and the signal receiver 230 to obtain height data of the subject, processes data transmitted by the weighing and body analyzing mechanism 213 to obtain weight and body compositions data of the subject, and transmits the height data and the weight and body compositions data to the display screen 216. The display screen 216 can display the height data and the weight and body compositions data transmitted by the signal processor 215. The signal processor 215 further transmits the height data and the weight and body compositions data to the external mobile device or server for storage via the wireless communication device 214.

The aforementioned body analyzer includes the main body 210 and the detecting board 240 separated from the main body 210. The detecting board 240 is provided with the signal emitter 220. The main body 210 includes the signal receiver 230, the information transmitter 212, the weighing and body analyzing mechanism 213, the wireless communication device 214, the signal processor 215, and the display screen 216. The signal emitter 220 transmits the height measuring signal. The height measuring signal is received by the signal receiver 230. The wireless communication device 214 interacts with the external mobile device or server via the wireless network. The signal processor 215 is electrically connected to the signal receiver 230, the information transmitter 212, the weighing and body analyzing mechanism 213, the wireless communication device 214 and the display screen 216, respectively. The signal processor 215 processes the data of the signal emitter 220, the signal receiver 230, the weighing and body analyzing mechanism 213, and transmits the processed data to the display screen 216 for display and transmits the processed data to the external mobile device or server for storage via the wireless communication device 214. In this way, an accurate weight and height data of the subject can be obtain via the processing of the signal processor on the received signal, which avoids manually height measurement via mechanical means, and ensures an accuracy of a measuring result. The measuring result can be visually displayed on the display screen and can be transmitted to the external mobile device or server for storage via the wireless communication device, thereby avoiding manually record of the measuring result, which saves time and labor, and is convenient and practical.

It should be understood that according to design requirements, the signal receiver 230 can be located on the detecting board 240, while the signal emitter 220 can be located on the main body 210. When measuring, the subject steps on the platform 211 and holds the detecting board 240 abutting against the top head thereof, the signal transmitted by the signal emitter 220 can be received by the signal receiver 230, and a height composition can be obtained by calculating according to a propagation time and a propagation speed of the signal. The propagation speed of the signal is constant for a certain signal emitter 220.

Similarly, in alternative embodiment, the signal emitter 220 and the signal receiver 230 can be both located on the detecting board 240. When measuring, the subject steps on the platform 211 and holds the detecting board 240 abutting against the top head thereof. The signal emitter 220 transmits a height measuring signal. The height measuring signal is reflected by the platform 211. The reflected height measuring signal is received by the signal receiver 230. The height composition of the subject can be obtained by calculating according to a propagation time and a propagation speed of the signal. The propagation speed of the signal is constant for a certain signal emitter 220.

The technical features of the above embodiments can be arbitrarily combined. For the sake of brevity of description, all possible combinations of the technical features in the above embodiments are not described. However, as long as there is no collision in the combination of these technical features, it should be considered as the scope described in this specification.

The foregoing implementations are merely specific embodiments of the present disclosure, and are not intended to limit the protection scope of the present disclosure. It should be noted that any variation or replacement readily figured out by persons skilled in the art within the technical scope disclosed in the present disclosure shall all fall into the protection scope of the present disclosure. Therefore, the protection scope of the present disclosure shall be subject to the protection scope of the claims.

What is claimed is:

1. A body analyzer, comprising:
a main body comprising a platform in contact with feet of a subject;
a detecting board separated from the main body and configured to abut against a top head of the subject, the detecting board being capable of moving towards or away from the platform;
wherein at least one of the main body and the detecting board is provided with a signal emitter, at least one of the main body and the detecting board is provided with a signal receiver, the signal emitter and the signal receiver are configured to measure a distance between the platform and the detecting board; the main body further comprises an information transmitter, a weighing and body analyzing mechanism, a signal processor, and a wireless communication device; the information transmitter is configured to transmit data of the signal emitter and the signal receiver to the weighing and body analyzing mechanism; the weighing and body analyzing mechanism is configured to calculate body compositions of the subject; the signal processor is configured to process data from the signal emitter, the signal receiver, and the weighing and body analyzing mechanism; and the wireless communication device is configured to transmit a processed data to an external mobile device or server via a wireless network.

2. The body analyzer according to claim 1, further comprising an acoustic indicator device electrically connected to the signal processor, and configured to broadcast processed data transmitted from the signal processor in voice.

3. The body analyzer according to claim 1, wherein the main body further comprises a display screen connected to the signal processor and configured to receive and display processed data transmitted from the signal processor.

4. The body analyzer according to claim 1, wherein the signal emitter is at least one selected from the group consisting of a laser emitter, an LED emitter, an infrared emitter, an ultrasonic transmitter, an electromagnetic wave transmitter, and a millimeter wave transmitter.

5. The body analyzer according to claim 1, wherein the wireless communication device is at least one selected from the group consisting of: a Wi-Fi device, a Bluetooth device, a ZigBee device, a Zwave device, a WLAN device, a GSM device, a 2G device, a 3G device, a 4G device, and a 5G devices.

6. The body analyzer according to claim 1, further comprising a power supply device configured to supply power to the body analyzer.

7. The body analyzer according to claim 1, wherein the body composition is at least one selected from the group consisting of weight, body fat, body water, body muscle, basal metabolism, body mass index, bone mass, and other compositions.

8. The body analyzer according to claim 1, wherein the detecting board is a plastic plate or a cell phone.

9. A body analyzer, comprising:
a main body comprising a platform in contact with feet of a subject, wherein the main body is provided with a signal emitter and a signal receiver;
a detecting board separated from the main body and configured to abut against a top head of the subject, the detecting board being capable of moving towards or away from the platform, wherein the detecting board, the signal emitter, and a signal receiver cooperatively measure a distance between the platform and the detecting board;
wherein the main body further comprises a weighing and body analyzing mechanism, a signal processor, an information transmitter, a wireless communication device, and a display screen; the wireless communication device is configured to perform a data interaction with an external mobile device or server via a wireless network; the information transmitter is configured to transmit data of the signal emitter and the signal receiver to the weighing and body analyzing mechanism; the weighing and body analyzing mechanism is configured to calculate body compositions of the subject; the signal processor is electrically connected to the weighing and body analyzing mechanism, the signal emitter, the signal receiver, the wireless communication, and display screen respectively, and configured to process data of the signal emitter, the signal receiver, and the weighing and body analyzing mechanism, and a processed data is sent to the display screen for displaying and to the external mobile device or server via the wireless communication device.

10. The body analyzer according to claim 9, wherein the signal emitter is at least one selected from the group consisting of: a laser emitter, an LED emitter, an infrared emitter, an ultrasonic transmitter, an electromagnetic wave transmitter, and a millimeter wave transmitter.
